(19) United States
(12) Patent Application Publication
(10) Pub. No.: US 2007/0281347 A1
(43) Pub. Date: Dec. 6, 2007

(54) Device and method for automated transfer of small organisms

(75) Inventors: Matthew Boeckeler, Northborough, MA (US); Bryan Chavez, San Jose, CA (US); Christopher J. Cummings, Brookline, MA (US); Carol M. Singh, Somerville, MA (US); Thomas P. Low, Belmont, CA (US); Max Crittenden, Menlo Park, CA (US)

Correspondence Address: PALMER & DODGE, LLP;KATHLEEN M. WILLIAMS, 111 HUNTINGTON AVENUE, BOSTON, MA, 02199, US

(73) Assignee: Envivo Pharmaceuticals, Inc.

(21) Appl. No.: 11/510,521

(22) Filed: Aug. 25, 2006

Related U.S. Application Data
(60) Provisional application No. 60/714,070, filed on Sep. 2, 2005.

(51) Int. Cl. C12N 1/00

(52) U.S. Cl. 435/243; 435/307.1

(57) ABSTRACT

A device is disclosed for transferring an organism from a donor container to a recipient container. The device comprises a transfer plate assembly comprising a first subassembly and a second subassembly. The transfer plate assembly positions donor containers and recipient containers in an inverted relationship so that the organism pass from the donor container to the recipient container. In addition, a method is presented for transferring a small organism from a donor container to a recipient container. The method includes engaging a donor container to a transfer plate assembly. The method discloses engaging a recipient container to the transfer plate assembly wherein the recipient container is in an inverted orientation to the donor container and rotating the transfer plate assembly so that the recipient container is positioned below the donor container which allows the small organism to be transferred.

### FIELD OF THE INVENTION

The invention relates to a device and a method for transferring small organisms. More specifically, the invention relates to a device and method for transferring a small organism from a donor container to a recipient container for further storage and/or analysis.

### BACKGROUND OF THE INVENTION

Genomics based drug discovery using model organisms such as Drosophila or Caenorhabditis holds great promise for the pharmaceutical industry. However, the advent of this experimental approach has only accentuated the problems associated with the maintenance and manipulation of large numbers of invertebrate organisms over often-prolonged periods of time. For example, several centers around the world maintain large libraries of Drosophila mutants. These lines are used for genetic screens and target mining for drug discovery. As no protocol currently exists to date for cryostorage of Drosophila, these organisms must be continually transferred to new vials with fresh nutrients every 3 to 4 weeks. The traditional approach of manually transferring Drosophila from a donor container to a recipient container is an extremely time consuming and labor intensive task (requiring small repetitive movements) that can cause potential employee injury and rapid burn out.

As such, there is a need in the art for a method and device which allows for the automated transfer of small organisms.

### SUMMARY OF THE INVENTION

The present invention features a device for transferring a small organism (e.g., a fly) from a capped donor container to a recipient container. The device includes a frame and a transfer plate assembly engaged to the frame. The transfer plate assembly includes a first subassembly and a second subassembly that are capable of working together to transfer the organism. The subassemblies engage a donor container, uncap the donor container, and position a recipient container in an inverted position relative to the donor container. Next, the transfer plate assembly rotates the two containers so that the recipient is positioned below the donor container. This allows the small organism to pass to the recipient container. Once the small organism has been transferred to the recipient container, a new cap is inserted into the recipient container.

The device includes a mechanism to move a plurality of recipient containers and/or donor containers to and/or from the transfer plate assembly (where the organism is actually transferred). More specifically, the device includes an upper engaging assembly positioned above the transfer plate assembly and a lower engaging assembly positioned below the transfer plate assembly. The engaging assembly may be a lift pin assembly where each lift pin assembly has a plurality of pins that move in combination to move a container (donor or recipient) to or from the transfer plate assembly.

In addition, a method is presented for transferring a small organism from a donor container to a recipient container. The method includes engaging a donor container having a small organism to a transfer plate assembly. The transfer plate assembly includes a first subassembly and a second subassembly that are capable of working together to transfer the organism. The subassemblies engage a donor container, uncap the donor container, and position a recipient container in an inverted position relative to the donor container. The method includes engaging a recipient container to the transfer plate assembly wherein the recipient container is in an inverted orientation as compared to the donor container. Further, the method includes removing a used cap from the donor container and sliding the first subassembly in relation to the second subassembly so that the recipient container in positioned directly above the donor container. Next, the transfer plate assembly is rotated about 180 degrees so that the recipient container is positioned below the donor container, thereby allowing the small organism to be transferred from the donor container to the recipient container.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 shows a small organism transfer device.

FIG. 2 shows a view of the device of FIG. 1 wherein various elements have been removed for clarity.

FIG. 3 shows an engaging assembly.

FIG. 4 shows a transfer plate assembly.

FIG. 5 shows a first plate of the transfer plate assembly.

FIG. 6 shows a clip holder of the transfer plate assembly.

FIG. 7 shows a second plate of the transfer plate assembly.

FIG. 8 shows a cap puller of the transfer plate assembly.

FIG. 9A-FIG. 9H show an overview of a method for transferring a small organism from a donor container to a recipient container.

FIG. 10A-FIG. 10T show an additional overview of a method for transferring a small organism from a donor container to a recipient container.

While the above-identified drawings set forth certain embodiments of the present invention, other embodiments of the present invention are also contemplated, as noted in the discussion. This disclosure presents these illustrative embodiments by way of representation and not limitation. Numerous other modifications and embodiments can be devised by those skilled in the art which fall within the scope and spirit of the principles of the present invention.

### DETAILED DESCRIPTION

A device and a method are presented for transferring a small organism such as a fly from a capped donor container to a recipient container. The small organism may be transferred from the donor container to the recipient container for continued storage, for continued analysis of the small organism or for a variety of other reasons. The device may transfer a single small organism or the device may substantially simultaneously transfer any number of small organisms from any number of donor containers to any number of recipient containers.

The method may be automated which reduces worker injury (injury caused by such a repetitive process) and maintains a clean, uncontaminated environment. Further, the device and method allow for the transfer of a small organism from a donor container to a recipient container without the need for any outside agent (i.e., anesthesia) applied to the small organism. Anesthesia is not required because the transfer plate assembly maintains a closed environment for the small organism throughout the present method (i.e., the opening of the container which holds the small organism is always blocked by a cap, another container in an inverted position, or a component of the transfer plate assembly). As such, the small organism transfer device provides an efficient device and method wherein contamination from anesthesia, manual transfer, etc. is substantially eliminated.

The capped donor containers are moved to the transfer plate assembly by the use of engaging assemblies such as an upper lift pin assembly and a lower lift pin assembly. The transfer plate assembly is where the small organisms are transferred from the donor container to the recipient containers. Next, a recipient container is engaged to the transfer plate assembly so that the recipient container is in an inverted alignment with respect to the donor container and positioned above the donor container. The transfer plate assembly is capable of removing the used cap of the donor container and aligning the donor container directly beneath the recipient container. Next, the device rotates the transfer plate assembly approximately 180 degrees so that the recipient container is now positioned beneath the donor container. Rotating the assembly allows for the small organism to be easily transferred from the donor container to the recipient container. An additional force (e.g., a vibration) may be supplied to the donor container by the device to facilitate the transfer of the organism. Once the organism has been successfully transferred from the donor container to the recipient container, a new cap may be inserted into the recipient container and the recipient container (now containing the small organism) may be removed from the transfer plate assembly.

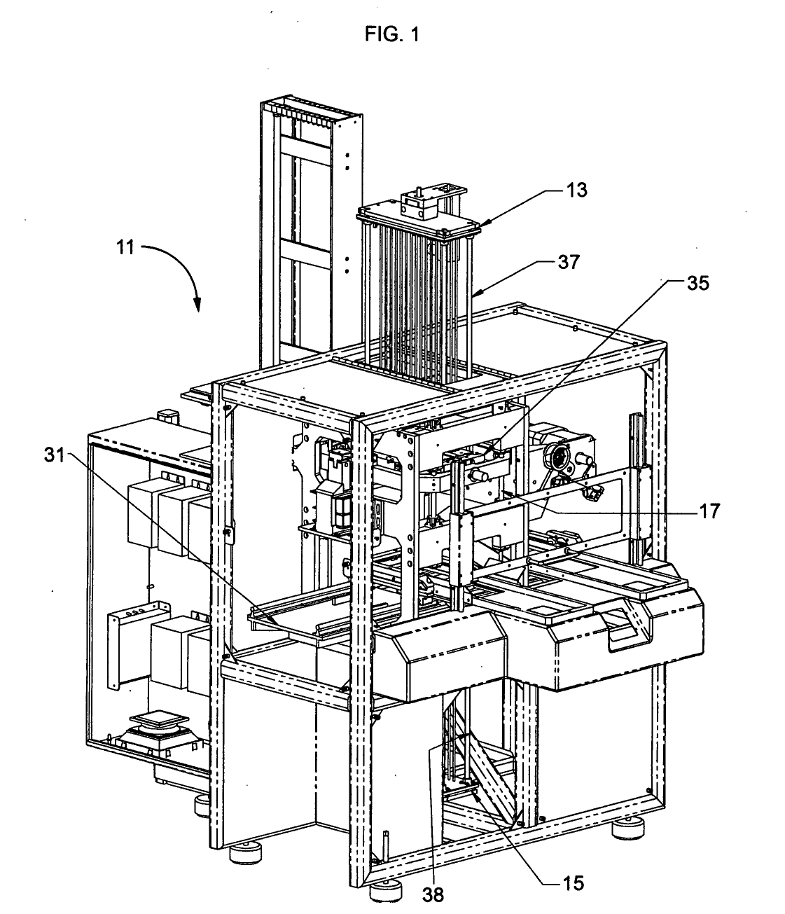

FIGS. 1-10 illustrate the device and method in greater detail.

FIG. 1 shows a small organism transfer device 11. The device 11 comprises an upper lift pin assembly 13 and a lower lift pin assembly 15. The upper lift pin assembly 13 is engaged to a first motor (not shown) and the lower lift pin assembly 15 is engaged to a second motor (not shown). Alternatively, the upper lift pin assembly 13 and the lower lift pin assembly 15 are engaged to the same motor (not shown). The upper lift pin assembly 13 and the lower lift pin assembly 15 are each capable of moving in a vertical direction. The motor provides the power for such movement in a vertical direction.

The upper lift pin assembly 13 includes a first set of pins 37 and the lower lift pin assembly 15 includes a second set of pins 38. There may be many pins 37, 38 from each lift pin assembly 13, 15 that are responsible for engaging a plurality of donor containers and/or recipient containers and delivering or removing the desired container to or from the transfer plate assembly 35. Those skilled in the art will recognize that the pins 37, 38 may engage and/or deliver the containers to the transfer plate assembly 35 in a variety of ways.

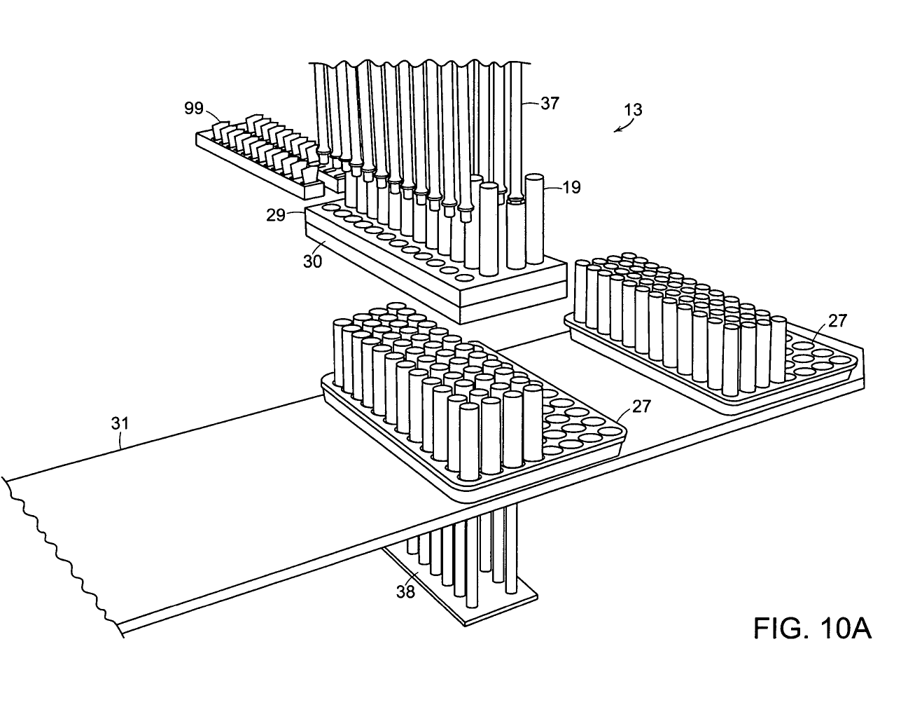

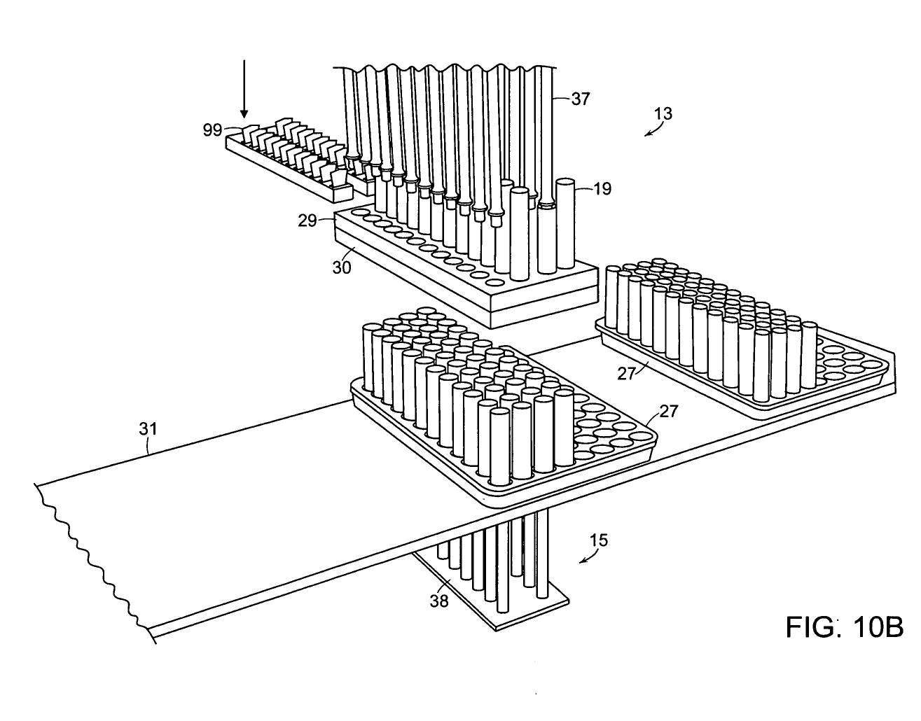

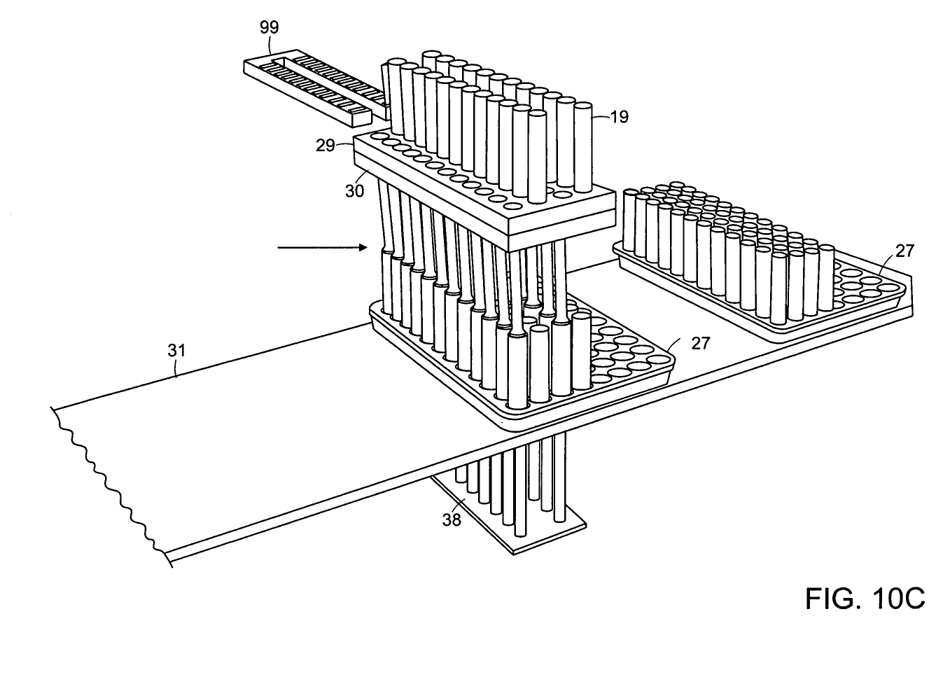

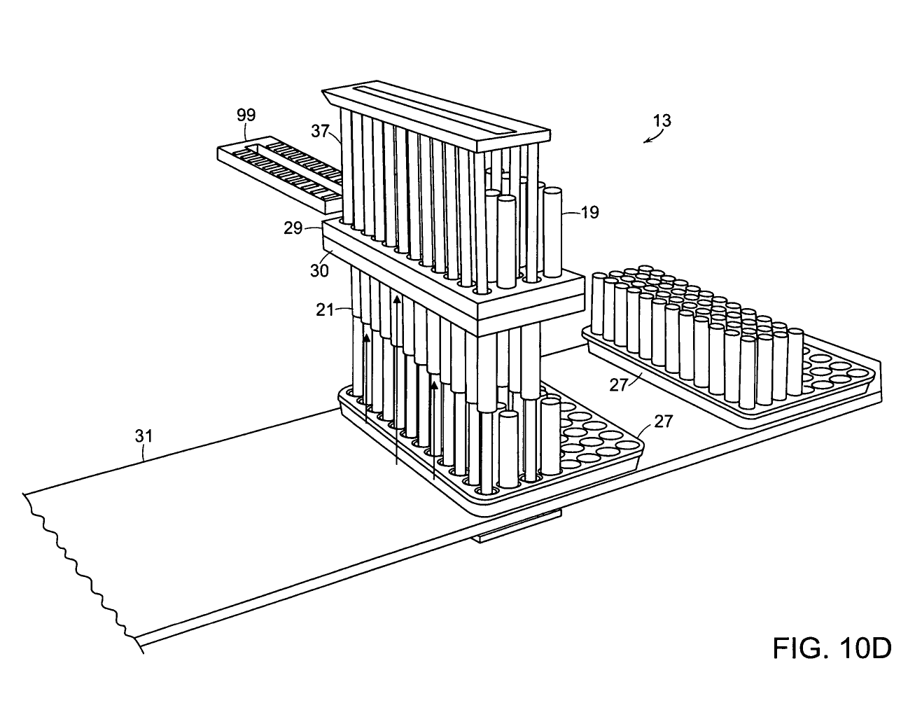

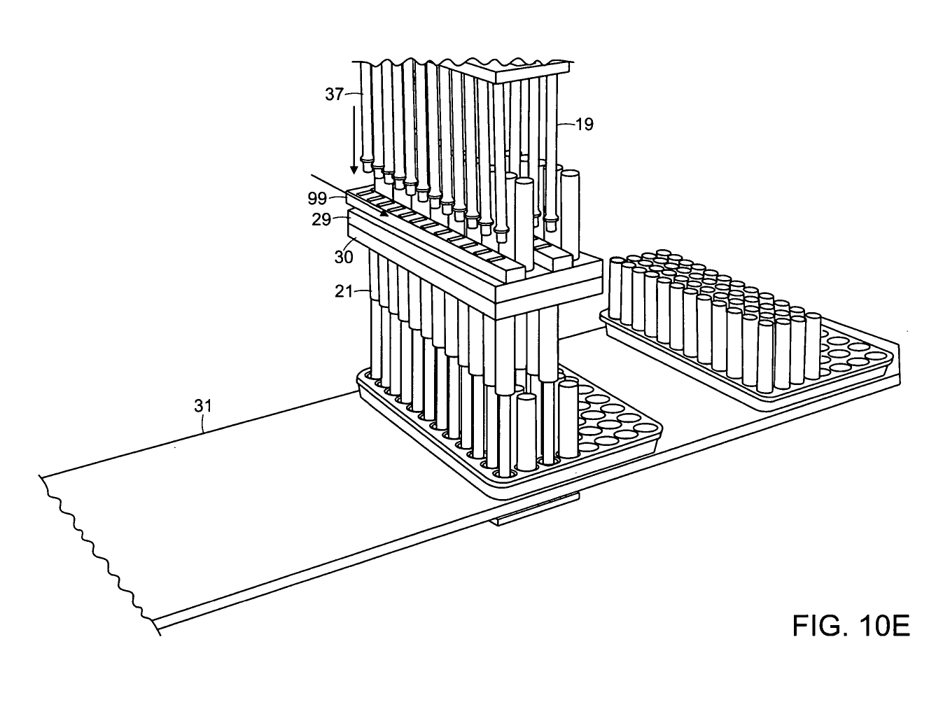

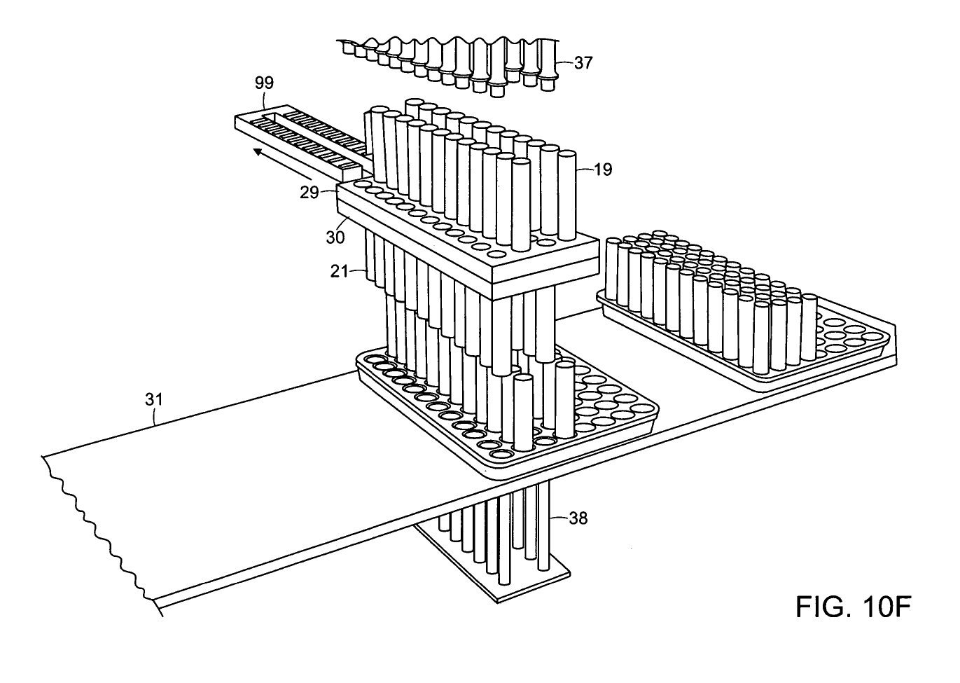

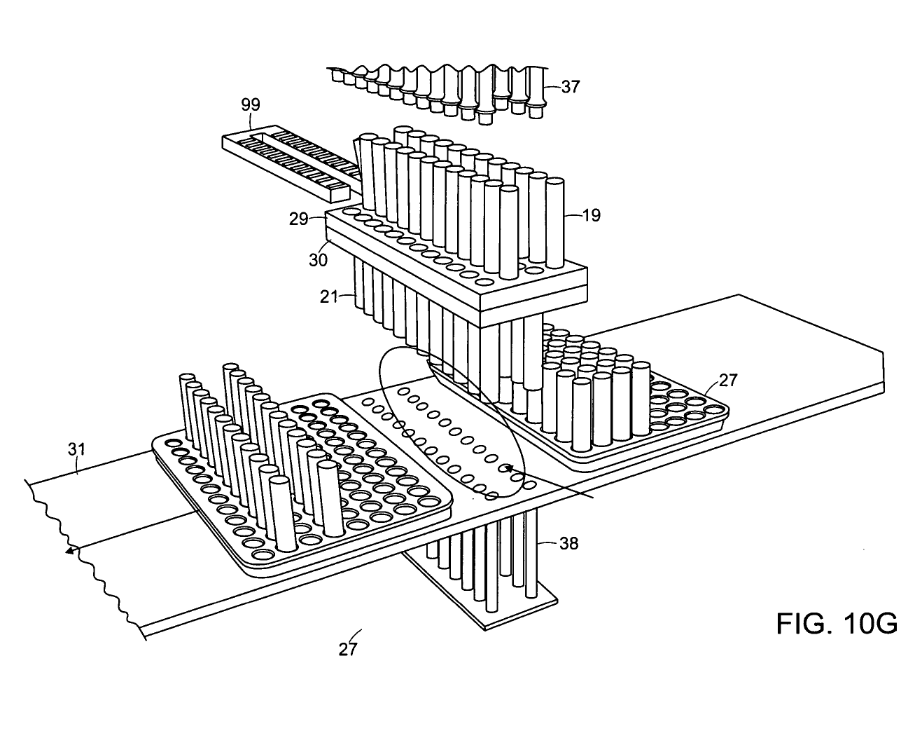

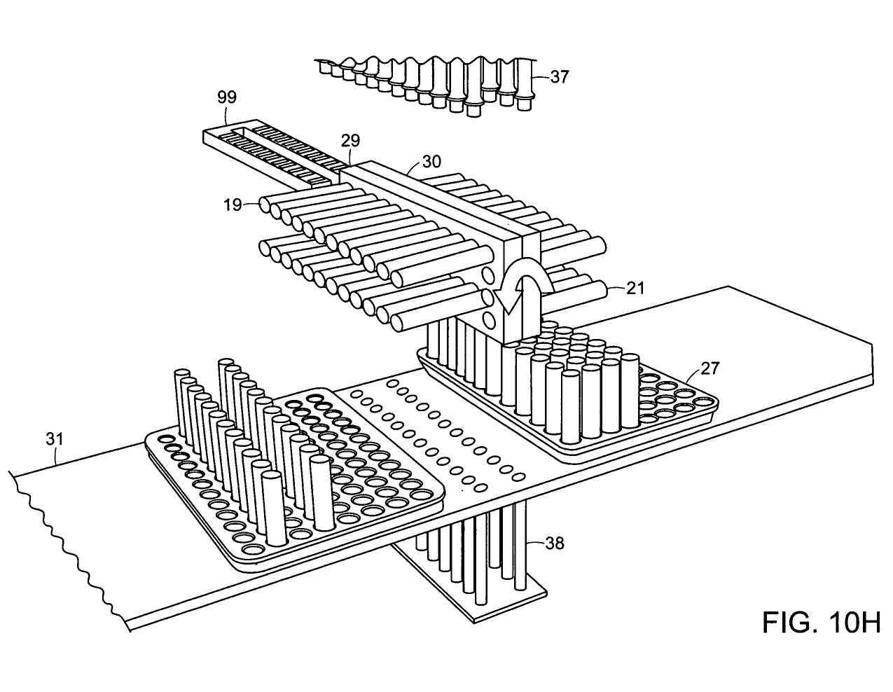

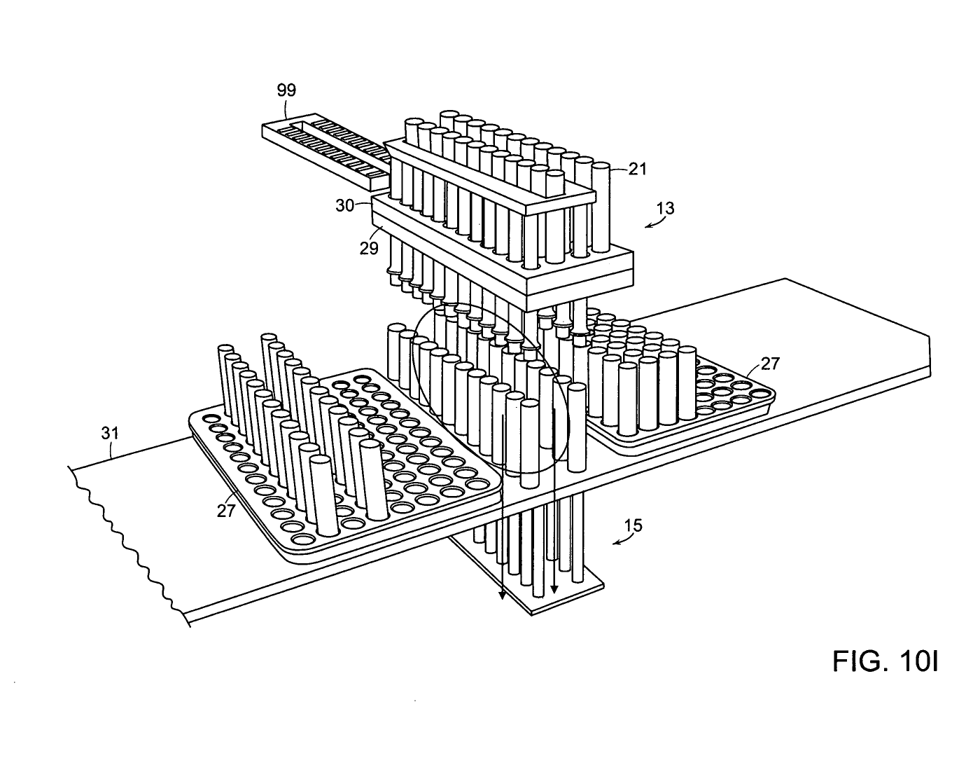

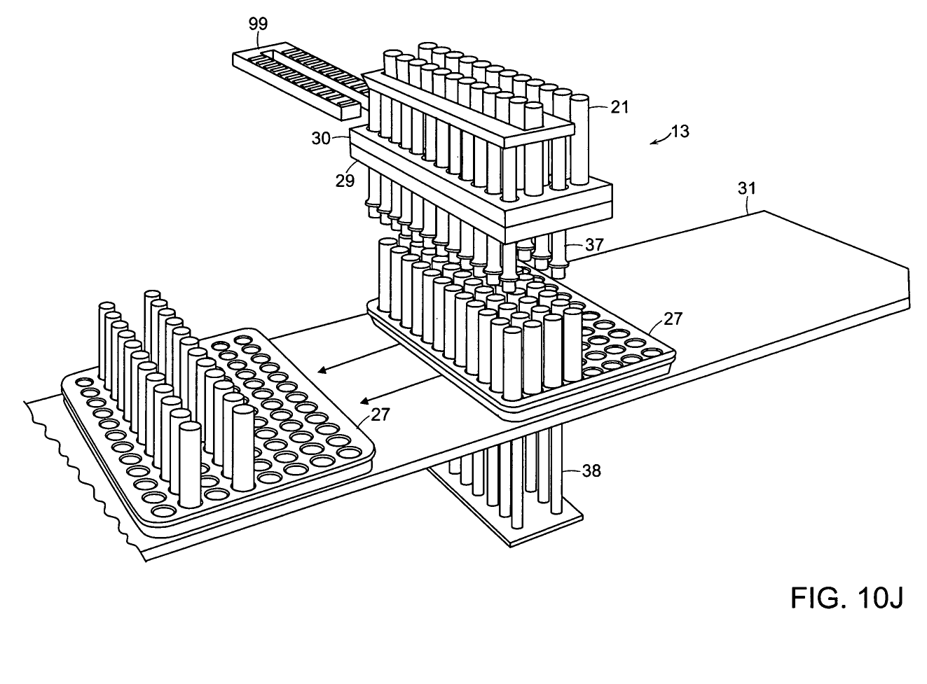

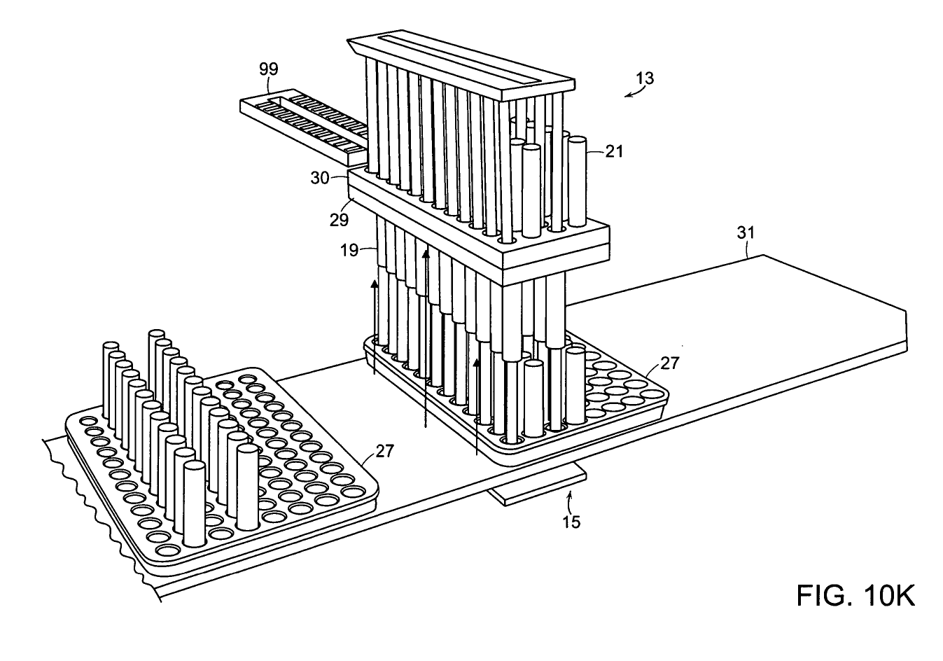

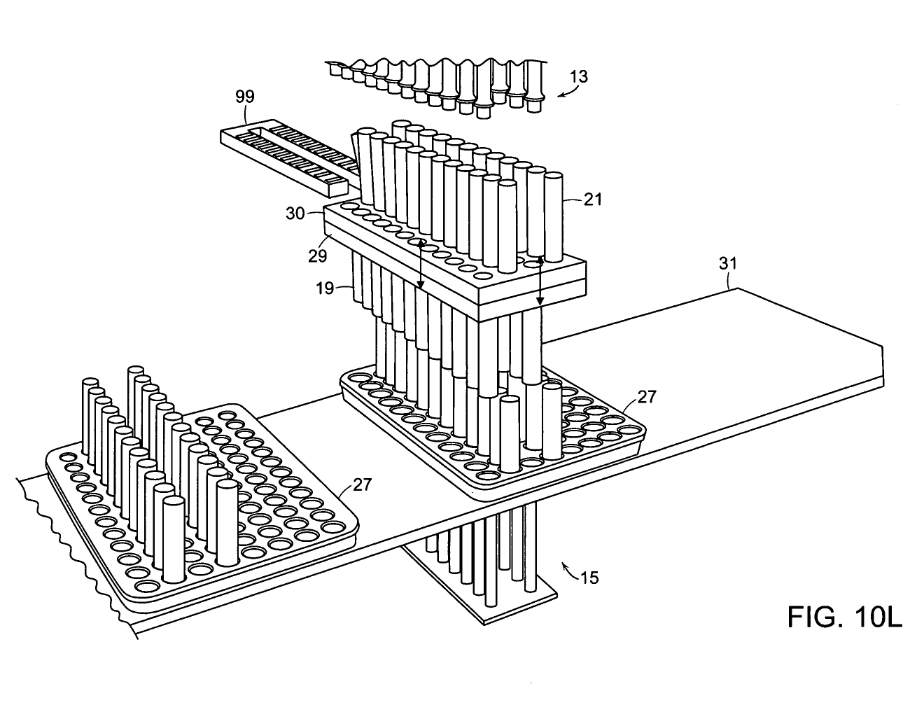

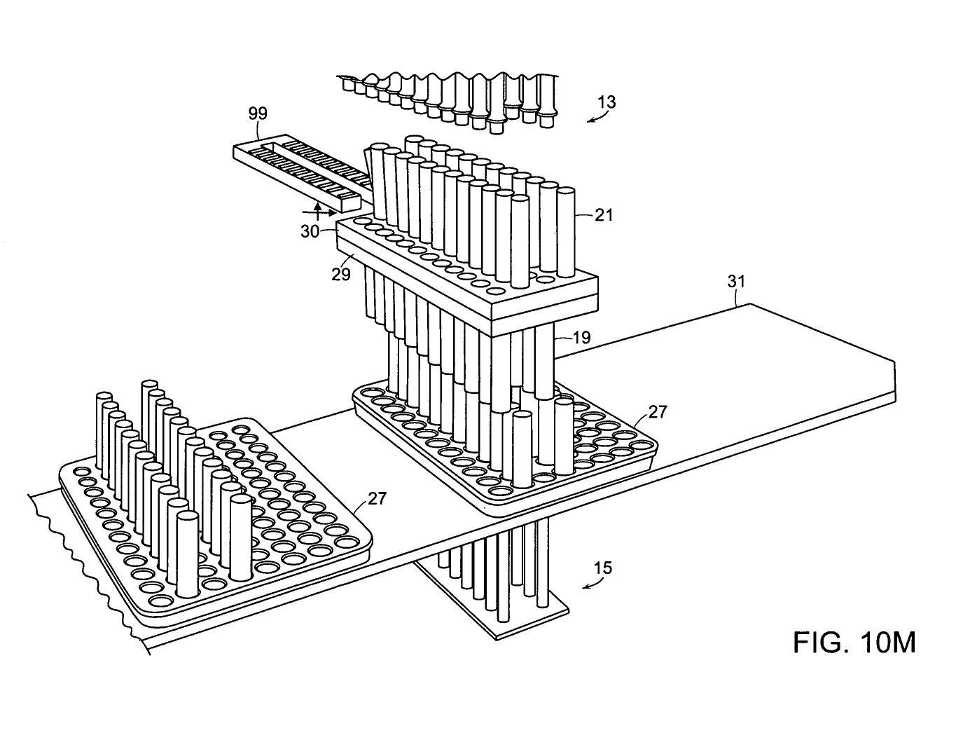

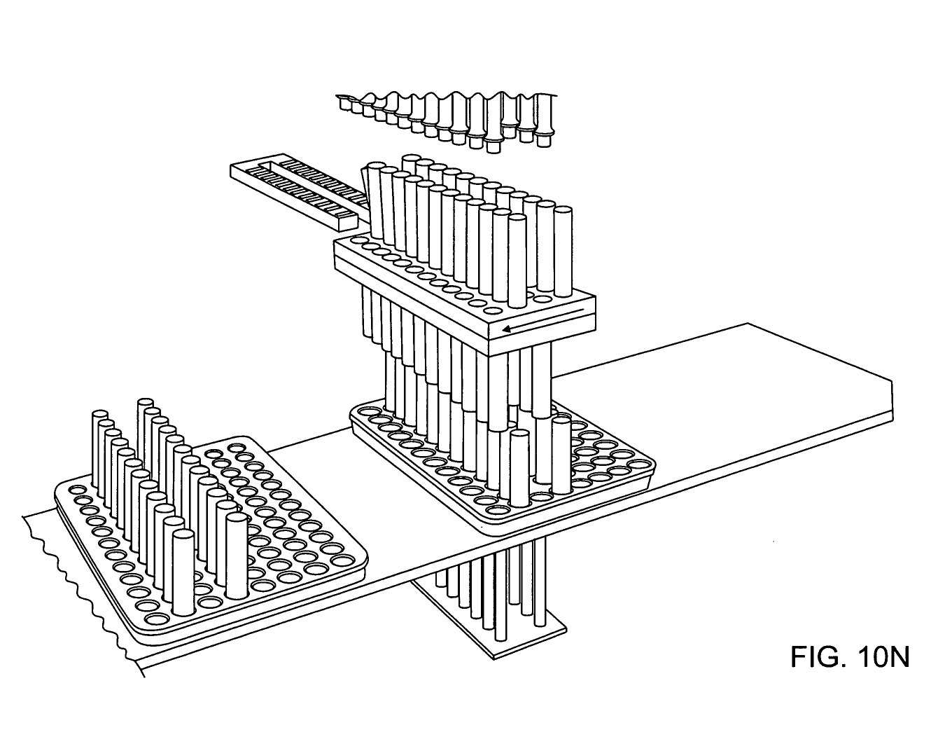

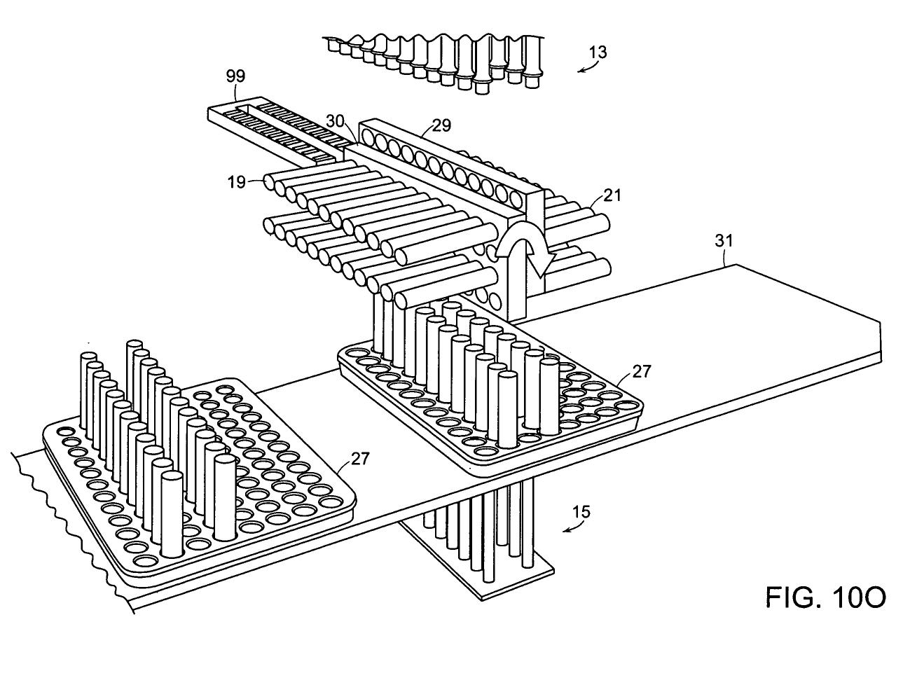

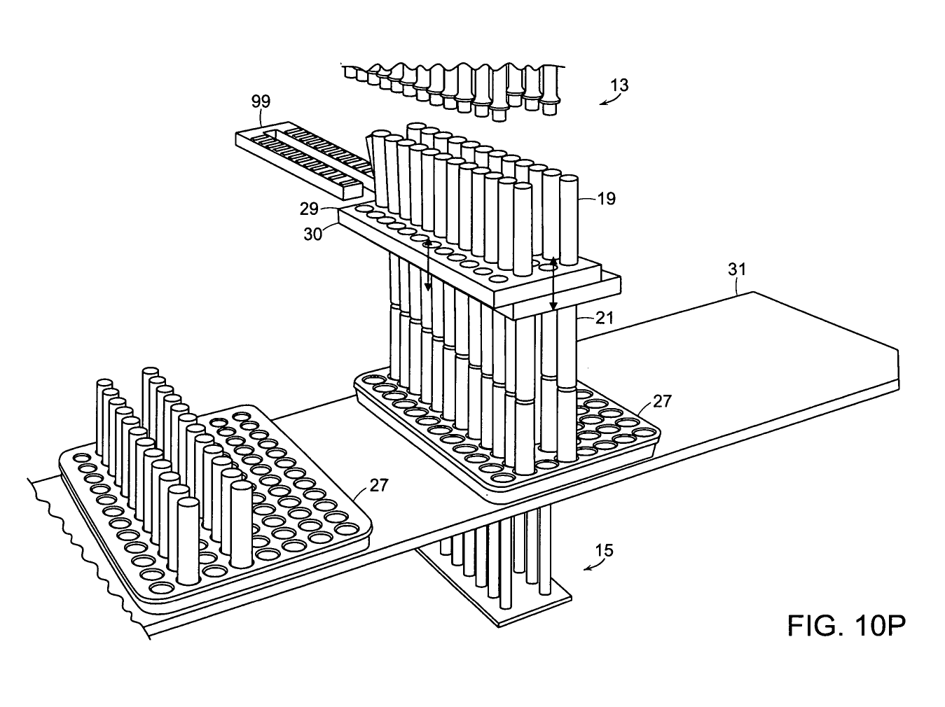

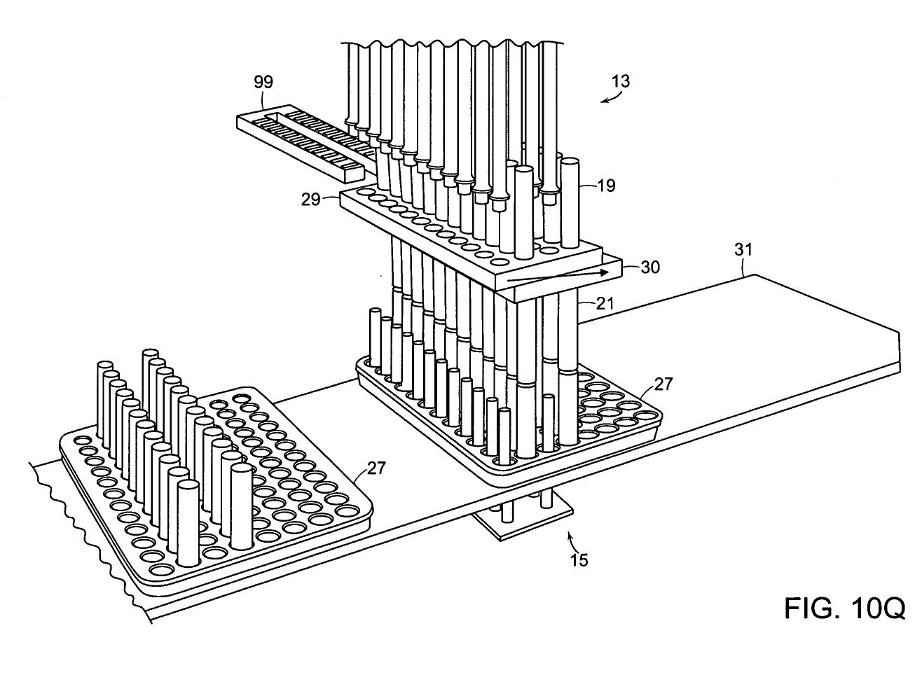

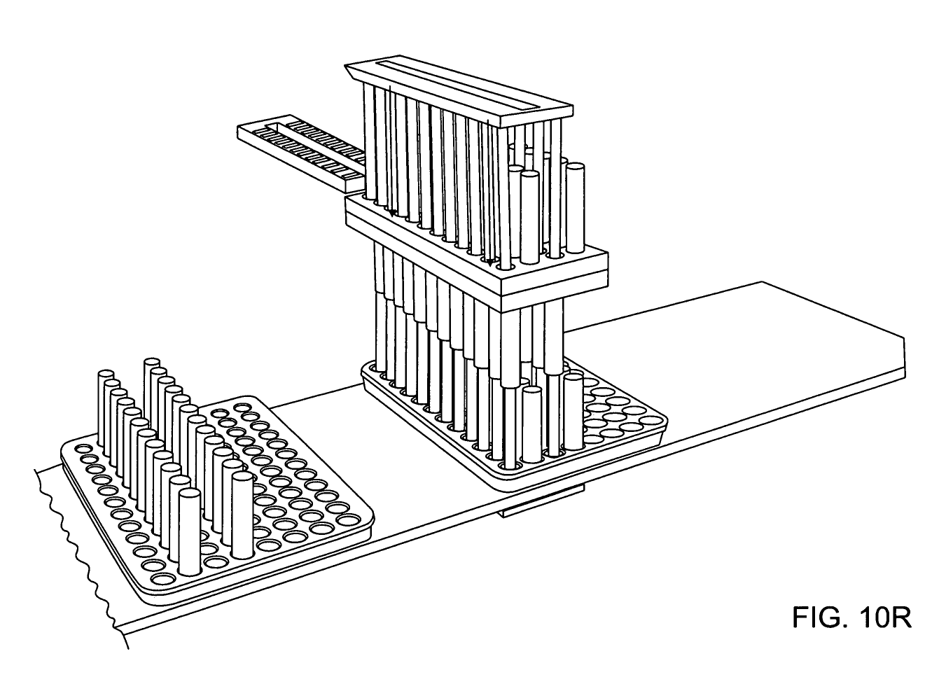

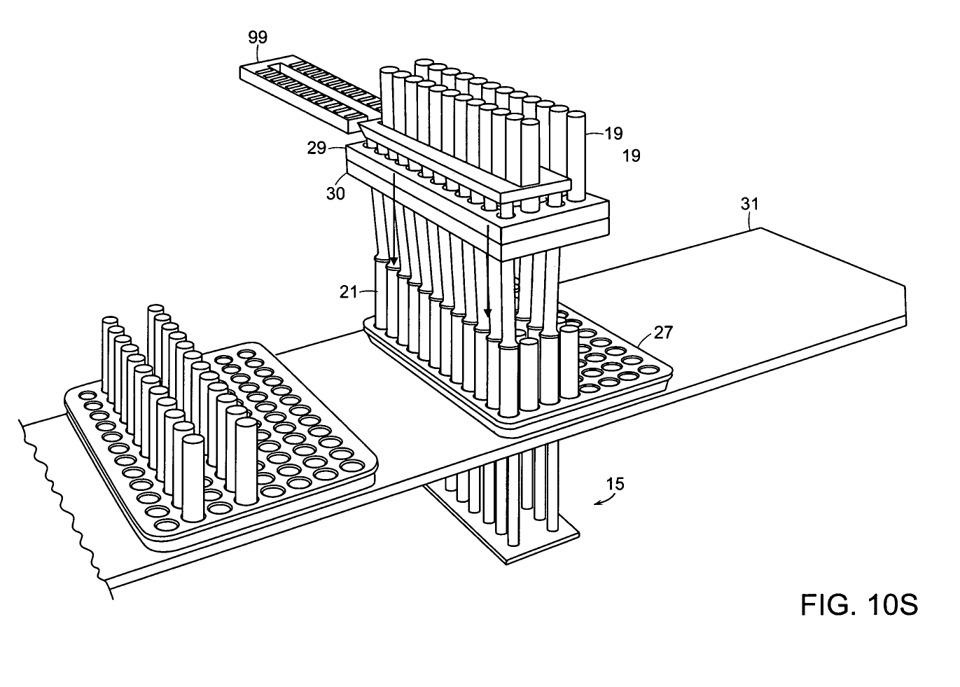

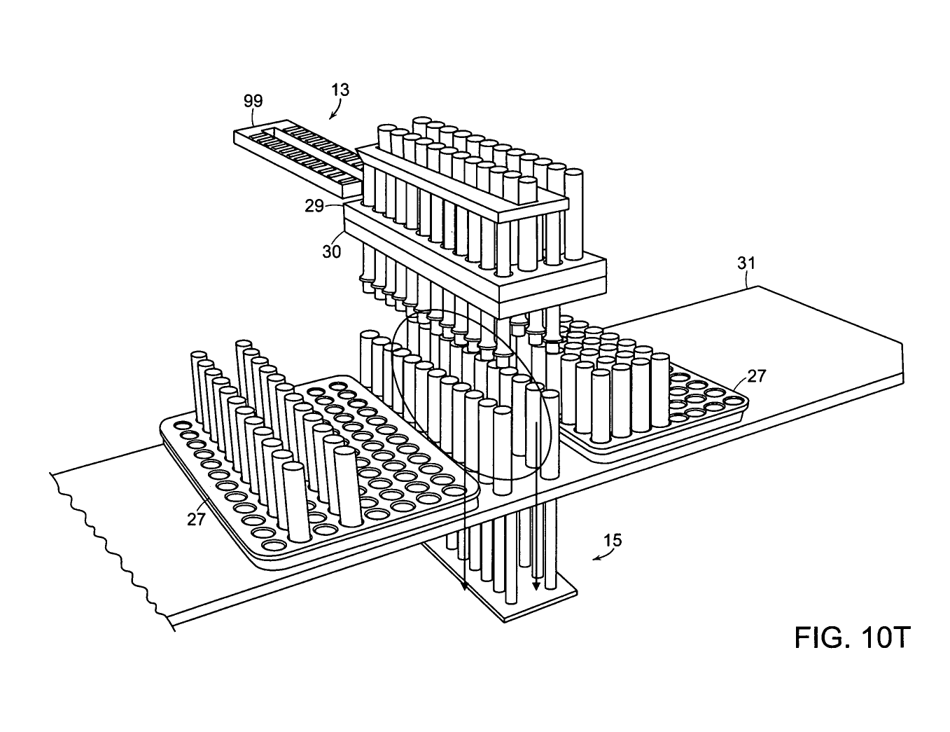

The small organism transfer device 11 includes a sliding shuttle 31. A plurality of racks (as shown in FIG. 10A-FIG. 10T) may be placed upon the sliding shuttle 31. A rack may hold a plurality of capped donor containers to be moved to the transfer plate assembly, or may hold a plurality of open recipient containers into which small organisms will be transferred. Additionally, once the small organism has been transferred from the donor container to the recipient container, a rack may be used to store the recipient containers which now contain the small organism. Alternatively, there may be multiple slide shuttles 31 or each sliding shuttle 31 may have multiple racks of donor and/or recipient containers.

The sliding shuttle 31 includes a plurality of holes and each rack has a corresponding set of holes. The plurality of holes in both the sliding shuttle and in each rack is of such a diameter as will allow a plurality of pins 38 from the lower lift pin assembly 15 to pass through the sliding shuttle 31 and then through the rack in order to engage a plurality of containers positioned in the rack. As stated above, allowing the plurality of pins 38 to engage the containers allows for the pins to deliver or remove the containers from the transfer plate assembly 35.

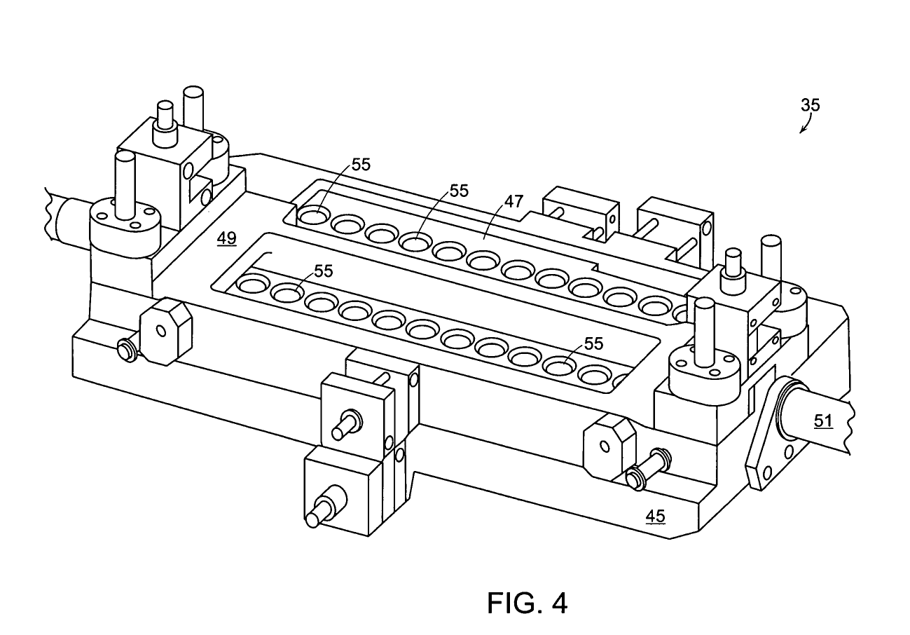

As shown in FIG. 1, the device 11 comprises a transfer plate assembly 35 which comprises a first subassembly and a second subassembly. As will be discussed in relation to FIG. 4-FIG. 8, the first and second subassemblies have various components which allow for the donor container to transfer the organism to the recipient container. These components restrain the donor container, allow for a used cap to be removed from the donor container, and position a recipient container above the donor container in an inverted position. Next, the transfer plate assembly rotates approximately 180 degrees allowing the small organism to “fall” into the recipient container. Following the successful transfer of a small organism to the recipient container, a new cap is inserted into the recipient container.

The device also includes a bump coil (solenoid) 17 engaged to the transfer plate assembly 35. The bump coil 17 delivers an action to the transfer plate assembly 35 to facilitate the transfer of an organism from a donor container to a recipient container. The action can include a short, rapid movement (i.e., a bump) or a vibration in order to facilitate a transfer of an organism from a donor container to a recipient container. Those skilled in the art will recognize that the bump coil 17 may supply any of a variety of actions to the transfer plate assembly 35 in order to facilitate the transfer of an organism from a donor container to a recipient container.

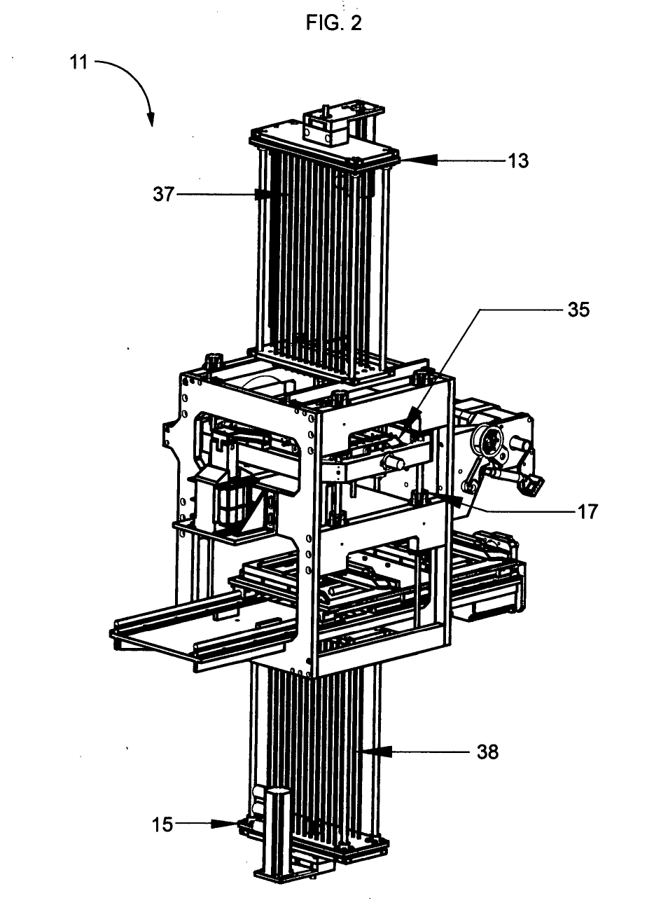

FIG. 2 shows an alternative view of the device as shown in FIG. 1. In FIG. 2, the relationship between the upper lift pin assembly 13, lower lift pin assembly 15, pins 37, 38, sliding shuttle 11, bump coil 17 and the transfer plate assembly 35 is more clearly shown.

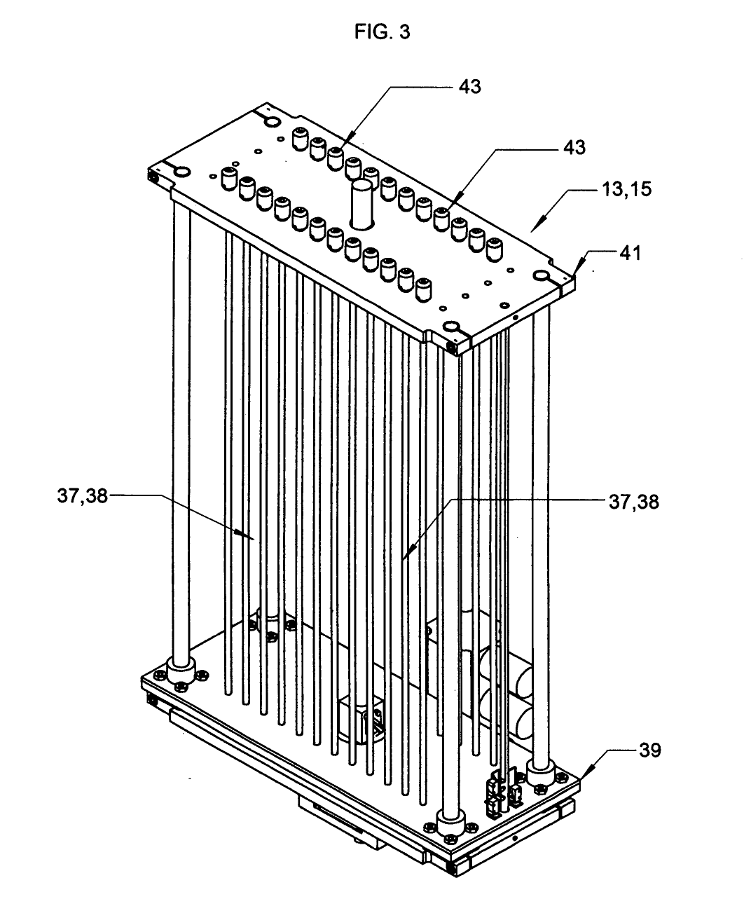

FIG. 3 shows a lift pin assembly such as can be used for the upper lift pin assembly 13 or lower lift pin assembly 15. As shown, the lift pin assembly 13, 15 includes 24 pins 37, 38. The lift pin assembly 13, 15 may include any number of pins 37, 38 and remain within the spirit and scope of the present invention.

The lift pin assembly 13, 15 includes a first moving plate 39 and a second stationary plate 41. The first moving plate 39 moves vertically towards the second stationary plate 41 (which remains stationary) so that the plurality of pins 37, 38 extend beyond the second stationary plate 41. The plurality of pins 37, 38 are extended beyond the second stationary plate 41 in order to engage a donor container and/or a recipient container. Those skilled in the art will recognize that a variety of engaging assemblies may be utilized in order to engage a plurality of containers with a plurality of pins. For example, any device capable of engaging a plurality of containers and moving the containers along a first axis to a transfer plate assembly wherein the transfer plate assembly is oriented substantially perpendicular to the first axis so that the containers are allowed to pass through a plurality of wells (i.e., openings) of the transfer plate assembly is within the spirit and scope of the present invention.

A pin cap 43 is engaged to an end of at least one pin 37, 38. The pin cap 43 engages a donor container and/or a recipient container when the pins 37, 38 are extended towards the containers. The pin cap 43 facilitates the ability of the pin 37, 38 to engage and move a donor container and/or a recipient container. A pin cap 43 may be designed in a variety of forms in order to facilitate the ability of a pin 37, 38 to engage and move a donor container and/or a recipient container.

The pins 37 of the upper lift pin assembly 13 engage a container from a first end (i.e., top) of the container and the pins 38 of the lower lift pin assembly 15 engage a container from an opposite end (i.e., bottom). As such, the pins 37 of the upper lift pin assembly 13 and the pins 38 from the lower lift pin assembly 15 may be used in combination to deliver a container to a desired vertical location. Such a combination is used to deliver or remove a plurality of donor containers and/or recipient containers to or from the transfer plate assembly 35.

FIG. 4 shows a transfer plate assembly 35. As stated above, the transfer plate assembly comprises a first subassembly and a second subassembly. The first subassembly and the second subassembly are substantially parallel to one another and are slidable relative to one another. The first subassembly includes a first plate 45. The second subassembly includes a second plate 47 and a cap puller 49. Each of these components includes a plurality of wells 55. As used herein, a well 55 is a hole in a component of such a diameter that a container (donor or recipient) may pass completely through the well 55. As will be discussed, each component includes a clip holder 57 which is capable of being moved to an engaged position and thereby engaging a plurality of clips to a container and retaining the container in a desired position in the well 55.

The transfer plate assembly 35 includes a rotation rod 51 which allows for the transfer plate assembly 35 to be rotated approximately 180 degrees. Rotating the transfer plate assembly 35 allows for a small organism to “fall” from a donor container into a recipient container. A motor (not shown) delivers the force to rotate the transfer plate assembly. The transfer assembly plate 35 is rotated about an axis which is substantially perpendicular to the direction of movement of the upper and lower engaging assemblies 13, 15.

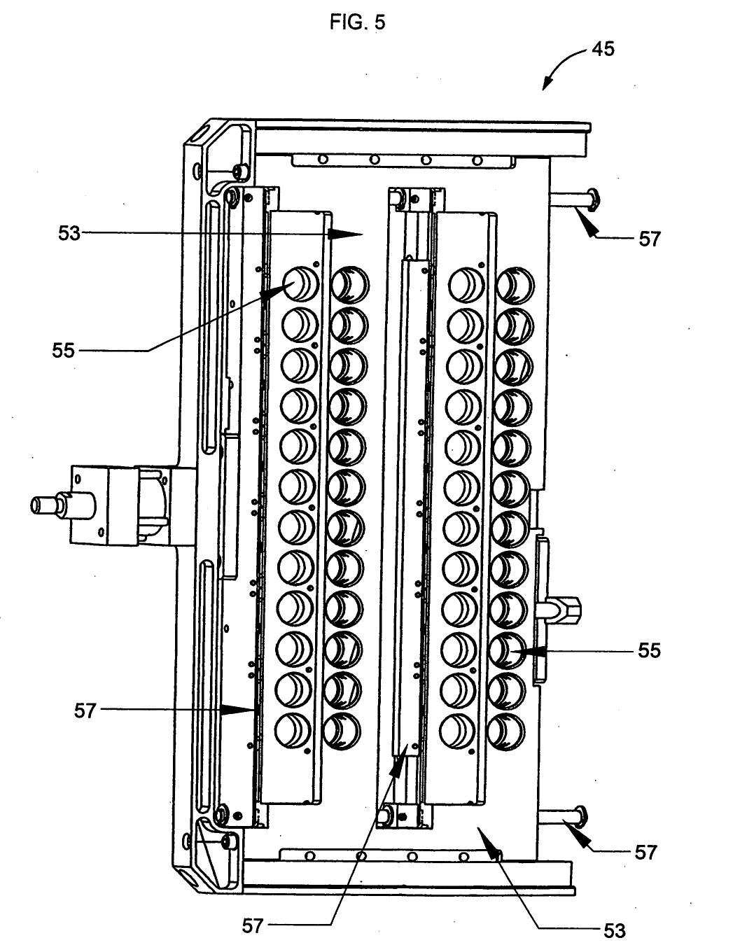

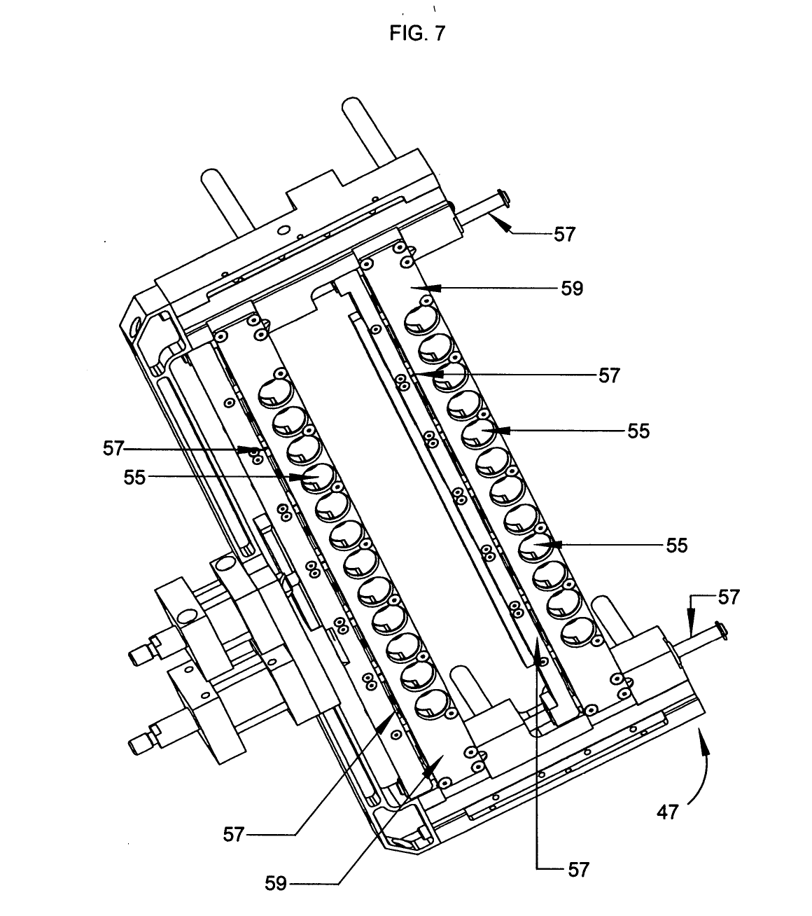

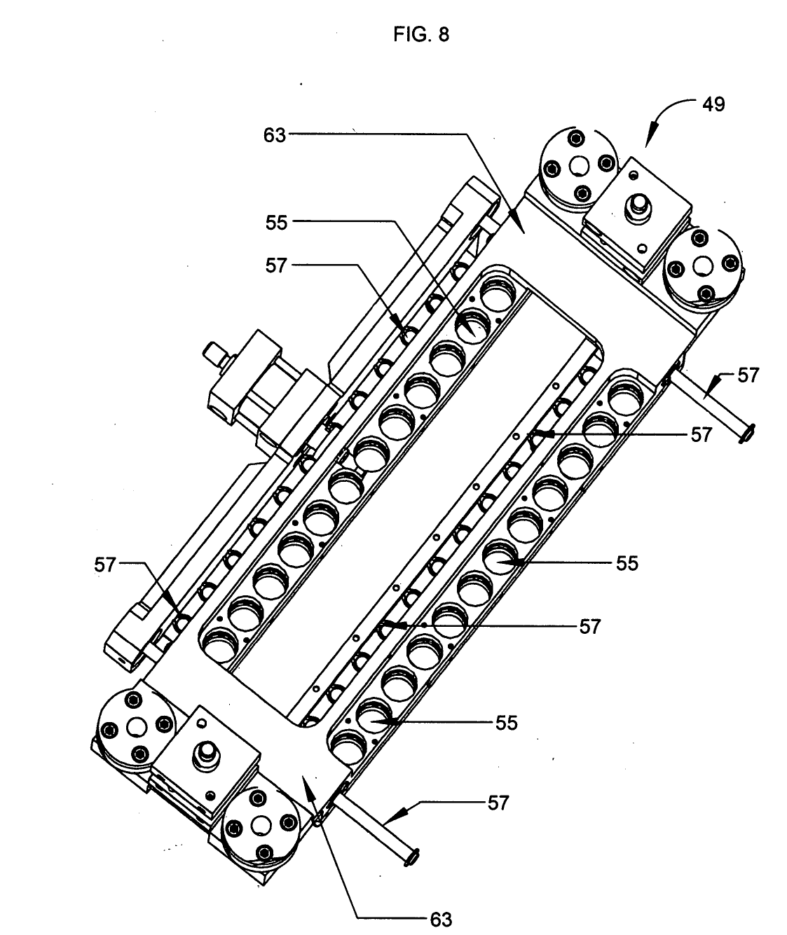

The first plate 45, the second plate 47 and the cap puller 49 each are individual components which each include various rows of wells 55. As seen in FIG. 5, the first plate 45 comprises 4 rows of 12 wells 55 per row; as seen in FIG. 7, the second plate 47 comprises 2 rows of 12 wells 55 per row; and as seen in FIG. 8, the cap puller 49 comprises 2 rows of 12 wells 55 per row. These three components are put together in such a manner to provide a transfer plate assembly 35 comprising 4 rows of 12 wells 55 each.

As shown in FIG. 5, the first plate 45 comprises 48 wells 55. The 48-wells 55 include four rows of 12 wells 55 per row. As shown in FIG. 7, the second plate 47 includes 24 wells 55. The 24 wells 55 of the second plate 47 includes a first row of 12 wells 55 and a second row of 12 wells 55. In assembling the transfer plate assembly 35, the second plate 47 is placed above the first plate 45 so that the wells 55 of the second plate 47 are aligned with a first row of 12 wells 55 of the first plate 45 and a third row of 12-wells 55 of the first plate 45.

Next, the cap puller 49 is placed on top of the second plate 47 so that the first and second rows of 12 wells 55 of the cap puller 49 are aligned with the second and fourth rows of 12 wells 55 of the first plate 45.

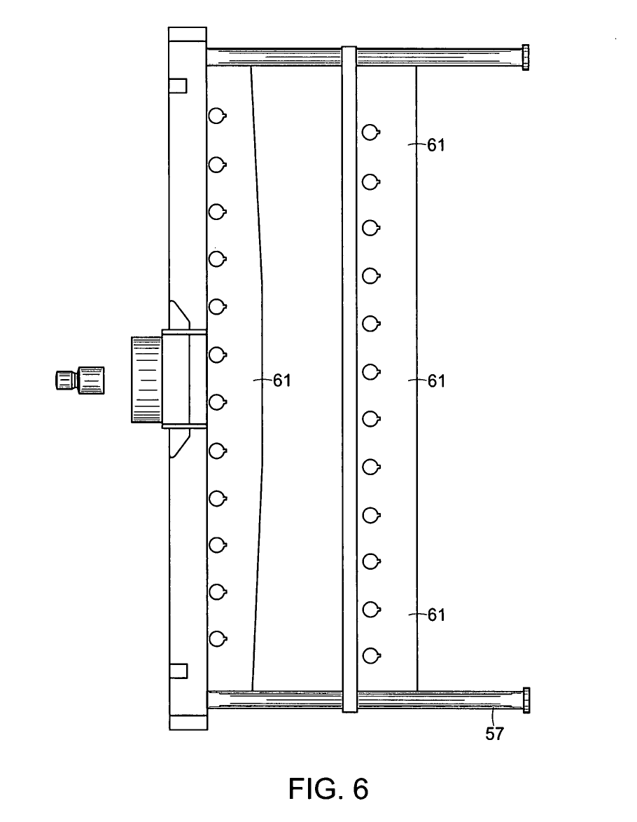

The clip holder 57 (not clearly shown in FIG. 5; see FIG. 6) is engaged to the first plate 45. The clip holder 57 may shift relative to the first plate 45 from an engaged position to a disengaged position. The clip holder 57 may engage a container once the container is positioned in a well 55; engaging a container restricts the container to the well 55.

FIG. 6 shows the clip holder 57. The clip holder 57 includes a plurality of clips 61. As described above in relation to FIG. 5, the first plate 45 includes a clip holder 57. The plurality of clips 61 are positioned so that the clips 61 may engage a container placed in a well 55. The clips 61 engage a container by adjusting the clip holder 57 from a disengaged position to an engaged position (a position wherein the clips 61 are engaging a container in the well 55). The clips 61 restrict a container to a well 55. The container may be removed from a well 55 when the clip holder 57 is positioned in a disengaged position (i.e., the clips 61 are retracted).

FIG. 7 shows a second plate 47. The second plate 47 includes 24-wells 55. A second clip holder 57 is engaged to the second plate 47. The clip holder 57 is engaged to the 24 well plate so that the clip holder 57 may engage a container entering a well 55 of the second plate 47 and therefore restrict a container to the respective well 55 of the second plate 47.

FIG. 8 shows the cap puller 49. A cap puller 49 includes 24 wells 55. The cap puller 49 includes a first row of 12 wells 55 and a second row of 12 wells 55. The cap puller assembly 49 includes a third clip holder 57. As such, the clip holder 57 may be positioned in an engaged position wherein the clip holder 57 restricts a container to a well 55 (as described above) or the clip holder 57 may be positioned in a disengaged position wherein the slip holder 57 does not engage a container and the container may be removed from a well 55.

The above-described device allows for an efficient transfer of organisms from a capped donor container to a recipient container. In addition, the invention presents a method for delivering a plurality of donor containers and/or recipient containers to or from the transfer plate assembly in order to transfer a plurality of small organisms (e.g., flies) from the various donor containers to the various recipient containers.

According to the method, the plurality of donor containers 19 are delivered to a first row of wells 55 of the transfer plate assembly 35. In addition, a plurality of donor containers 19 are delivered to the third row of wells 55 of the transfer plate assembly 35. Donor containers 19 (oriented cap up) pass through the first row of wells 55 in the first plate 45 and the used caps 23 (used caps 23 being those that cover donor containers 19) are engaged by a clip holder 57 in the first row of wells 55 of the cap puller 49. The donor containers 19 are engaged to the cap puller 49 by shifting a clip holder 57 of the cap puller 49 from the disengaged position to the engaged position once the used caps 23 enter the wells 55 of the cap puller 49. Further, a second set of capped donor containers 19 are similarly passed through the third row of wells 55 in the first plate 45 and are engaged by the clip holder 57 in the second row of wells 55 of the cap puller 49.

Next, a set of recipient containers 21 are engaged in an inverted position to a first row of wells 55 of the second plate 47. Next, a second set of recipient containers 21 are engaged in an inverted position to a second row of wells 55 of the second plate 47. As such, the recipient containers 21 are oriented in an inverted position above the donor containers 19.

Further, a first set of new caps 25 are retained in a second row of wells 55 of the first plate 45. In addition, a second set of new caps 25 are retained in a forth row of wells of the first plate 45. The new caps 21 are initially positioned directly beneath the recipient containers 21.

Next, the used caps 23 are removed from the donor containers 19 by applying a downward action to the donor containers 19 via the pins 38 of the lower lift pin assembly 15 while the used caps 23 continue to be engaged by the transfer plate assembly 35. Once the used caps 23 are removed from the donor container 19, the first subassembly 29 is slid relative to the second subassembly 30 so that the now uncapped donor containers 19 are positioned beneath inverted recipient containers 21.

The transfer plate assembly 35 is now rotated about 180 degrees so that the donor container 19 is now directly above the recipient container 21. This alignment facilitates the transfer of a small organism from the donor container 19 to the recipient container 21. The present method allows for an action of the bump coil 17 (shown in FIG. 1) to be applied to the transfer plate assembly 35 in order to facilitate this transfer. The action may be a vibration, a short, rapid motion (i.e., a bump), etc. Those skilled in the art will recognize that any force which facilitates the transfer of a small organism from a donor container 19 to a recipient container 21 is within the spirit and scope of the present invention.

Once the transfer is substantially complete, the first subassembly 29 is slid back to the original alignment relative to the second subassembly 30. The recipient containers 21 (now comprising the small organism) are now located directly beneath a respective new cap 25. Next, a plurality of pins 37 from the upper lift pin assembly 13 engage the new caps 25 and insert the new caps 25 into the recipient containers 21 by applying a downward force to the new caps 25.

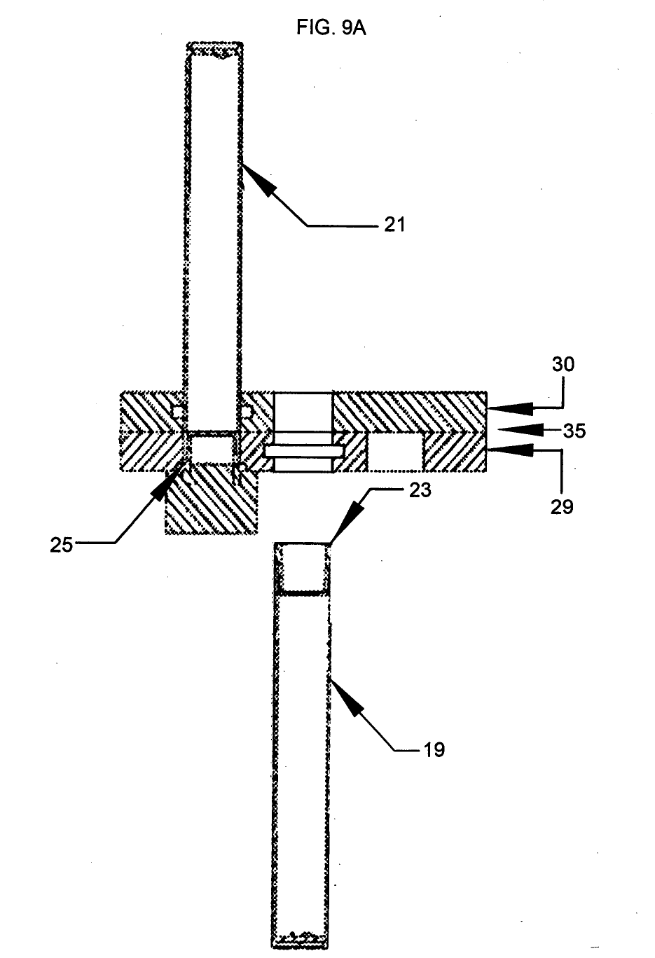

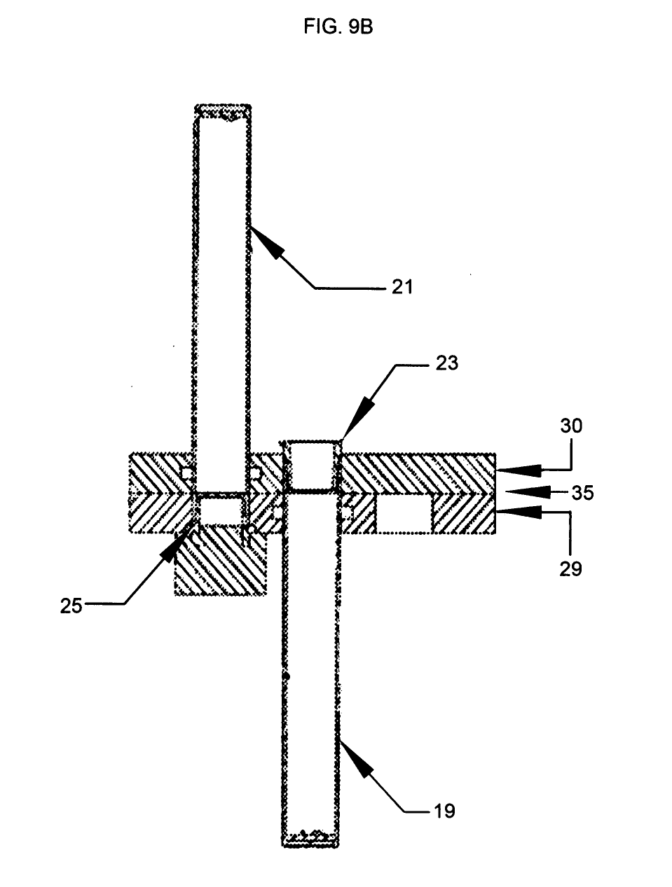

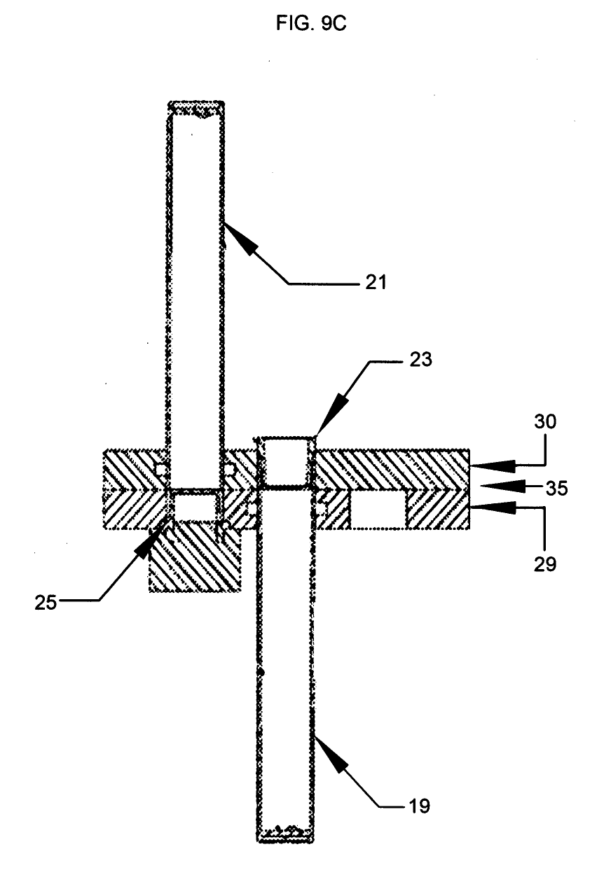

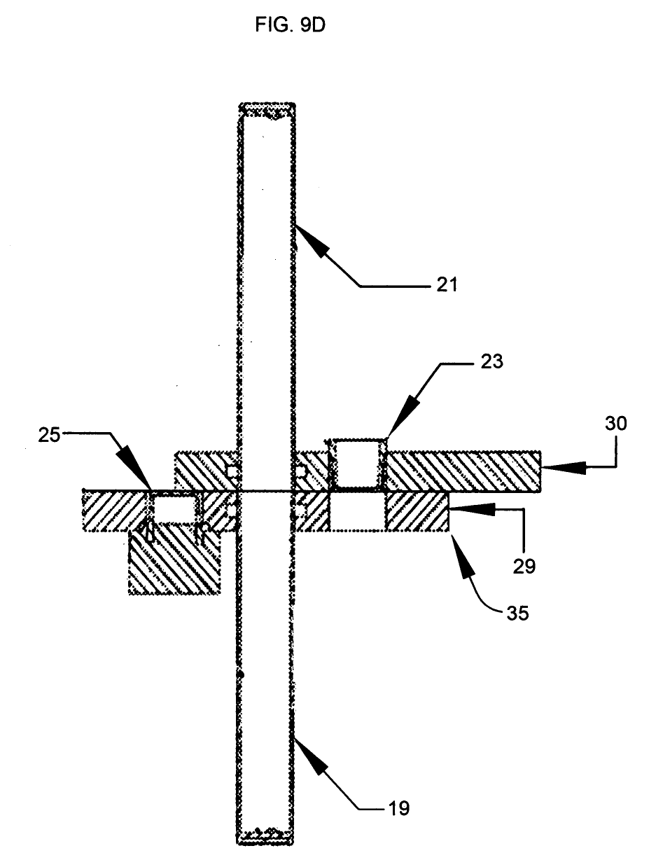

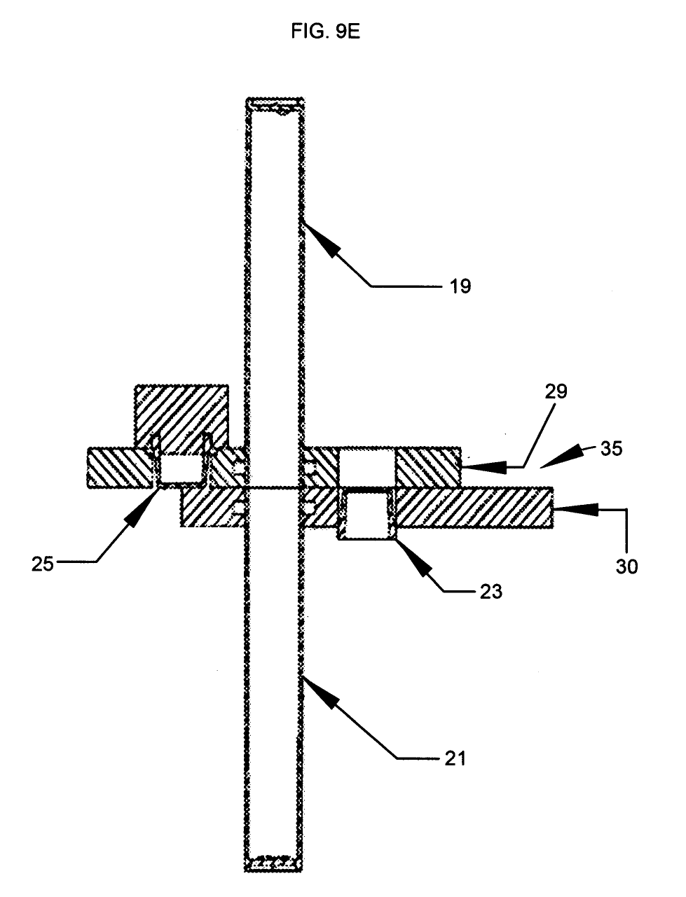

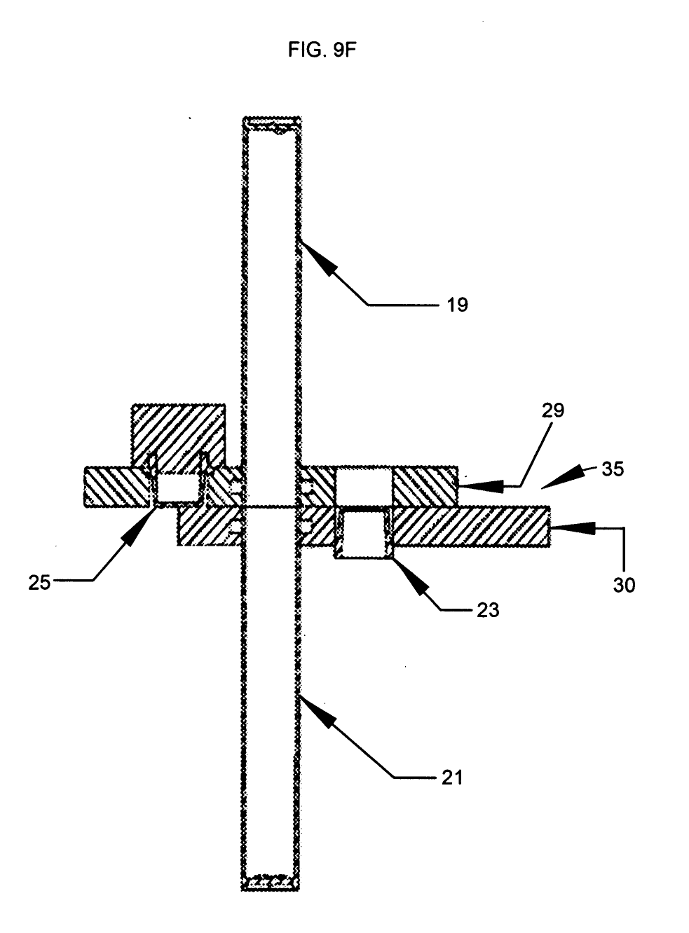

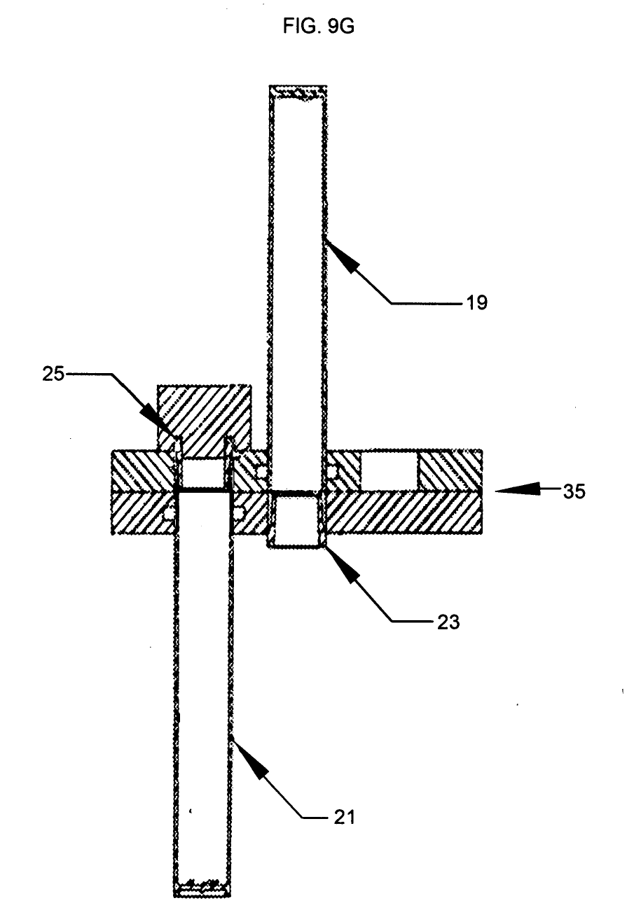

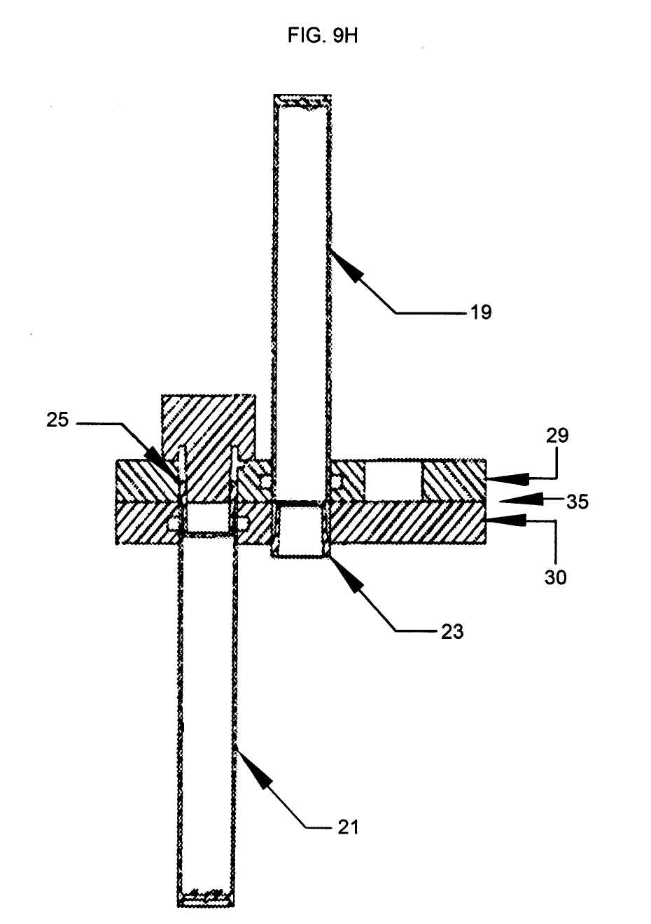

FIG. 9A-FIG. 9H presents an overview of a method which utilizes the small organism transfer device 11. FIG. 9A shows a recipient container 21 engaged to a transfer plate assembly 35. In addition, FIG. 9A shows a new cap 25 positioned in the transfer plate assembly 35 in order to deliver the new caps 25 into recipient containers once organisms have been transferred into the recipient containers. The new cap 25 is aligned substantially opposite the recipient container 21. FIG. 9A shows a donor container 19 having a small organism wherein the donor container 19 is closed by a used cap 23 (thereby confining the small organism to the donor container and/or preventing any potential contamination).

FIG. 9B shows the donor container 19 now moved to and engaged by the transfer plate assembly 35. More specifically, the donor container 19 passes through the first subassembly 29 and is engaged by a second subassembly 30 wherein the used cap 23 of the donor container 19 is clipped and retained by the second subassembly 30. As such, the used cap 23 may be disengaged from its donor container 19 by applying a downward force to the donor container 19 (wherein the used cap 23 remains engaged to the second subassembly 30 and the donor container 19 is lowered so that its opening is now aligned with the interface of the first subassembly 29 and the second subassembly 30). The downward force is supplied to the donor container 19 by a pin 38 of the lower lift pin assembly 15.

FIG. 9C shows the used cap 23 being engaged by the second subassembly 30 of the transfer plate assembly 35. Further, the donor container 19 is subjected to a downward force (while the used cap 23 remains engaged to the second subassembly 30) in order to disengage the donor container 19 from the used cap 23. The downward force may be applied by engaging a pin cap 43 to a container wherein when the lower engaging assembly is capable of “pulling” the donor container 19 in a downward direction while the used cap 23 remains engaged to the transfer plate assembly 35. The donor container 19 is pulled down to a position wherein the top of the donor container 19 is substantially linear with the interface between the second subassembly 30 and the first subassembly 29. Because the donor container 19 is aligned with the interface of the first subassembly 29 and the second subassembly 30 the small organism may not escape from the donor container; such an alignment eliminates the need for outside agents (such as anesthesia) to retain the small organism to the donor container 19 prior to transfer.

FIG. 9D shows the second subassembly 30 of the transfer plate assembly 35 slid relative to the first subassembly 29 of the transfer plate assembly 35 in order to align the now uncapped donor container 19 with the recipient container 21.

FIG. 9E shows the transfer plate assembly 35 rotated about 180 degrees. As such, the donor container 19 is now above the recipient container 21. Rotating the transfer plate assembly 35 allows for the small organism to “fall” from the uncapped donor container 19 (now inverted) to the recipient container 21.

FIG. 9F shows an action applied to the transfer plate assembly 35 from the bump coil 17 (shown in FIG. 1) in order to facilitate the transfer of an organism from the donor container 19 to the recipient container 21. The action may be a vibration applied to the transfer plate assembly 35. Alternatively, the action may be a short, rapid motion (i.e., a bump) applied to the transfer plate assembly 35. Those skilled in the art will recognize that various actions may be applied to the transfer plate assembly 35 to assist in the transfer of a small organism from the donor container to the recipient container and remain within the spirit and scope of the present invention.

Following the application of the above-described force to the transfer plate assembly 35, the small organism will have been transferred from the donor container 19 to the recipient container 21.

FIG. 9G shows the second subassembly 30 of the transfer plate assembly 35 slid relative to the first subassembly 29 of the transfer plate assembly 35 in order to realign the recipient container 21 with the new cap 25. The new cap 25 is inserted into the recipient container 21. As such, the recipient container 21, now having the small organism from the donor container 19, may be disengaged from the transfer plate assembly 35 and the donor container 19 and used cap 23 may be discarded.

The above-described method may be automated. In addition, the method allows for a small organism(s) from a plurality of donor containers 19 to be substantially simultaneously transferred from the plurality of donor containers 19 to a plurality of recipient container 21. Those skilled in the art will recognize that the contents of any number of donor containers 19 may be substantially simultaneously transferred to any number of recipient containers 21 and remain within the spirit and scope of the present invention.

FIGS. 10A-10T show an additional overview of the small organism transfer device 11. These figures illustrate the use of the pins 37 from the upper lift pin assembly 13 and pins 38 from the lower lift pin assembly 15 to move the containers to and/or from the transfer plate assembly 35.

FIG. 10A shows a transfer plate assembly 35 which includes a first subassembly 29 and a second subassembly 30. As shown, the first subassembly 29 includes a plurality of wells 55 and the second subassembly 30 includes a plurality of wells 55. The wells 55 in the first subassembly 29 and the wells 55 of the second subassembly 30 are of such a diameter as to allow a container to pass through the wells 55 (unless engaged by a clip holder 57).

FIG. 10A shows a plurality of pins 37, 38 capable of engaging the plurality of donor containers 19 and/or recipient containers 21. The plurality of pins 37, 38 engage the donor container 19 and/or the recipient container 21 and deliver the container to the transfer assembly plate 35 and/or remove the containers from transfer plate assembly 35.

FIG. 10A shows a sliding shuttle 31 wherein a first 96-container rack 27 containing a plurality of recipient containers 21 and a second rack 27 containing a plurality of donor containers 19. The sliding shuttle 31 comprises a plurality of holes. The holes of the sliding shuttle 21 allow for a plurality of pins 38 to pass through the sliding shuttle 31, engage a plurality of containers and deliver the containers to and/or from the transfer plate assembly 35.

FIG. 10A shows a cap loader 99 which may deliver a plurality of new caps 25 to the transfer plate assembly 35 wherein the new caps 25 are later inserted into the recipient containers 21. Those skilled in the art will recognize that new caps 25 may be delivered to the transfer plate assembly 35 by a variety of methods and remain within the spirit and scope of the present invention.

FIG. 10B shows a plurality of new caps 25 placed into the cap loader 99. As shown, the transfer plate assembly 35 of FIG. 10B shows a plurality of donor containers 19 engaged to the transfer plate assembly 35. As such, FIG. 10B illustrates shows a stage in the method wherein a plurality of organisms have already been transferred from the donor containers 19 to a plurality of recipient containers 21. The recipient containers 21 (now capped) have been removed from the transfer plate assembly 35. Therefore, as shown, the transfer plate assembly 35 is now ready to accept a new set of recipient containers 21, rotate the transfer plate assembly 35 approximately 180 degrees, discard the now empty donor containers 19, accept a new set of donor containers 19 (comprising organisms to be transferred), rotate the transfer plate assembly 35 approximately 180 degrees and transfer the contents of the donor container 19 to the recipient container 21. These various steps will be described in relation to FIG. 10C-FIG. 10T.

FIG. 10C shows a plurality of pins 37, 38 engaging empty recipient containers 21. The pins 37, 38 engage the recipient container 21 from the top and the bottom.

FIG. 10D shows a plurality of pins 37 delivering a plurality of recipient containers 21 to the transfer plate assembly 35.

FIG. 10E shows a cap loader 99 delivering a plurality of new caps 25 to the transfer plate assembly 35. The plurality of new caps 25 are delivered to a position opposite the recipient container 21 (i.e., the opening of the recipient container 21 is positioned beneath the new cap 25). FIG. 10F shows the cap loader 99 being retracted from the transfer plate assembly 35.

FIG. 10G shows a rack 27 containing a plurality of recipient containers 21 wherein the rack 27 may be moved on the sliding shuttle 31. A plurality of holes in the sliding shuttle 31 are now positioned beneath the transfer plate assembly 35. A plurality of holes in the rack 37 are aligned with the holes of the sliding shuttle 31. The holes in the sliding shuttle 31 allow for empty donor containers 19 to be removed and discarded from the transfer plate assembly 35. In addition, the holes in the sliding shuttle 31 and the holes in each rack 27 allow for pins 38 to pass through the sliding shuttle to engage containers and move containers relative to the transfer plate assembly 35.

FIG. 10H shows the transfer plate assembly 35 being rotated approximately 180 degrees. Once the transfer plate assembly 35 is rotated, the empty donor containers 19 are located on the below the empty recipient containers 21. FIG. 10I shows a plurality of pins 37, 38 moving the plurality of empty donor containers 19 from the transfer plate assembly 35. The empty donor containers 19 are discarded.

As such, the transfer plate assembly 35 is now in a similar step of the method as shown in FIG. 9A—the transfer plate assembly 35 is ready to accept a plurality of capped donor containers 19, remove the used cap 23 from the donor container 19, align the donor container 19 with the recipient container 21, rotate the transfer plate assembly 35 and subsequently transfer an organism from the donor container 19 to the recipient container 21. FIG. 10J-FIG. 10T illustrate this method.

FIG. 10J shows a rack 27 comprising a plurality of donor containers 19 (each of which comprises a small organism) positioned below the transfer plate assembly 35.

FIG. 10K shows a plurality of pins 37, 38 engaging the plurality of donor containers 37, 38. The plurality of pins 37, 38 engage the plurality of donor containers 19 from the top and from the bottom. The plurality of pins 37, 38 move the plurality of donor containers 19 to the transfer plate assembly 35. Those skilled in the art will recognize that various methods of delivering and/or removing the recipient 21 and donor containers 19 to and from the transfer plate assembly 35 are within the spirit and scope of the present invention.

FIG. 10L shows an action being applied to the transfer plate assembly 35 so that a small organism in a donor container 19 is now positioned at the bottom of the donor container 19. The action may be a vibration. Alternatively, the action may be a short, rapid motion (i.e., a bump). Applying such an action facilitates a later transfer step (i.e., make sure small organism is not stuck to an inner wall of the donor container 19).

FIG. 10M shows a plurality of used caps 23 of the donor containers 19 being engaged by the second subassembly 30 of the transfer plate assembly 35. The used caps 23 remain engaged by the second subassembly 30 while the donor containers 19 are pulled down so as to disengage from their respective used cap 23. The donor container 19 is pulled down so that the opening of the donor container 19 is aligned with the interface of the second subassembly 30 and the first subassembly 29 of the transfer plate assembly 35.

FIG. 10N shows the second subassembly 30 of the transfer plate assembly 35 being slid in relation to the first subassembly 29 of the transfer plate assembly 35 so that the opening of the recipient container 21 is directly above the opening (now uncapped) donor container 19.

FIG. 10O shows the transfer plate assembly 35 being rotated approximately 180 degrees so that the opening of the recipient container 21 is now directly below the opening of the donor container 19.

FIG. 10P shows an action being applied to the transfer plate assembly 35 in order to facilitate a transfer of a small organism from the donor container 19 to the recipient container 21. As stated above, the action may be a vibrational force. Alternatively, the action may be a short, rapid movement (i.e., a bump). Those skilled in the art will recognize that any type of force applied to the transfer plate assembly 35 in order to facilitate the transfer of an organism from a donor container 19 to a recipient container 21 is within the spirit and scope of the present invention.

FIG. 10Q shows a step wherein the first subassembly 29 of the transfer plate assembly 35 is slid relative to the second subassembly 30 of the transfer plate assembly 35 so that the new caps 25 (engaged by the first subassembly 29) are now positioned directly above the recipient containers 21 (which now comprise the small organism).

FIG. 10R shows a plurality of pins 37 engaged to the plurality of new caps 25 so that the new caps 25 are inserted into the recipient containers 21.

FIG. 10S shows a plurality of pins 37, 38 engaging the recipient containers 21 (now capped). The pins 37, 38 are utilized to disengage the recipient containers 21 from the transfer plate assembly 35. The recipient containers 21 (now containing the transferred small organism) are placed in a rack 27. As shown in FIG. 10T, the now empty donor containers 19 are discarded and the method is set to begin again.

FIG. 9A-FIG. 9H and FIG. 10A-10T are merely illustrative of the steps of a method which utilized the transfer device 11. Various steps may be omitted, repeated or added and the method would remain within the spirit and scope of the present invention.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A device for transferring a small organism from a donor container to a recipient container comprising:
a transfer plate assembly includes a first subassembly and a second subassembly wherein the transfer plate assembly engages a donor container in an inverted position relative to a recipient container;
a rotation rod engaged to the transfer plate assembly wherein the transfer plate assembly may be rotated about the rotation rod;
an upper engaging assembly engaged in a position above the transfer plate assembly; and
a lower lift pin assembly engaged in a position below the transfer plate assembly,
wherein the upper engaging assembly and the lower engaging assembly move in combination to move a container to or from the transfer plate assembly.

2. The device of claim 1 wherein the first subassembly comprises a first plate.

3. The device of claim 3 wherein the second subassembly comprises a second plate and a cap puller.

4. The device of claim 1 wherein the first subassembly is slidable in relation to the second subassembly.

5. The device of claim 1 further comprising a sliding shuttle located below the transfer plate assembly.

6. The device of claim 1 wherein the upper and lower engaging assemblies are upper and lift pin assemblies wherein each lift pin assembly includes a plurality of pins.

7. The device of claim 1 further comprising a bump axis assembly wherein the bump axis assembly is capable of providing a force to the transfer plate assembly in order to facilitate the transfer of a small organism from a donor container to a recipient container.

8. The device of claim 1 wherein the transfer plate assembly is capable of rotating approximately 180 degrees about an axis substantially perpendicular to a direction of movement of the upper and lower engaging assemblies.

9. A device for transferring a small organism from a donor container to a recipient container comprising:
a transfer plate assembly which includes a first subassembly and a second subassembly wherein the transfer plate assembly engages a donor container in an inverted position relative to a recipient container; and
a rotation rod engaged to the transfer plate assembly wherein the transfer plate assembly may be rotated about the rotation rod.

10. The device of claim 9 wherein the first subassembly includes a first plate having a plurality of wells.

11. The device of claim 9 wherein the second subassembly includes a second plate having a plurality of wells.

12. The device of claim 11 wherein the second subassembly includes a cap puller having a plurality of wells.

13. The device of claim 12 wherein the cap puller is engaged to a clip holder.

14. The device of claim 9 wherein the transfer plate assembly may be rotated approximately 180 degrees around the rotation rod.

15. A method of transferring a small organism from a donor container to a recipient container comprising:
engaging a donor container comprising a small organism to a transfer plate assembly wherein the transfer plate assembly includes a first subassembly and a second subassembly;
engaging a recipient container to the transfer plate assembly wherein the recipient container is in an inverted orientation as compared to the donor container;
removing a used cap from the donor container;
sliding the first subassembly in relation to the second subassembly so that the recipient container in positioned directly above the donor container; and
rotating the transfer plate assembly so that the recipient container is moved beneath the donor container which allows the small organism to be transferred from the donor container to the recipient container.

16. The method of claim 15 further comprising:
delivering a new cap to the first subassembly wherein the new cap may be inserted into a recipient container once the small organism has been transferred to the recipient container.

17. The method of claim 16 further comprising:
sliding the first subassembly in relation to the second subassembly so that the recipient container is positioned beneath the new cap.

18. The method of claim 17 further comprising:
inserting the new cap into the recipient container by applying a force to the new cap.

19. The method of claim 15 wherein a small organism from a plurality of donor containers are substantially simultaneously transferred to a plurality of recipient containers.

20. The method of claim 15 wherein the process is automated.

21. The method of claim 15 further comprising:
applying an action to the transfer plate assembly in order to facilitate the transfer of the small organism from the donor container to the recipient container.
